(19) United States
(12) Patent Application Publication
(10) Pub. No.: US 2009/0221054 A1
(43) Pub. Date: Sep. 3, 2009

(54) Biogas system

(76) Inventor: Bernhard Wett, Innsbruck (AT)

Correspondence Address: WENDEROTH, LIND & PONACK, L.L.P., 1030 15th Street, N.W.,, Suite 400 East, Washington, DC, 20005-1503, US

(21) Appl. No.: 12/382,550

(22) Filed: Mar. 18, 2009

Related U.S. Application Data
(63) Continuation of application No. PCT/AT2007/000423, filed on Sep. 6, 2007.

(30) Foreign Application Priority Data
Sep. 21, 2006 (AT) A 1578/2006

(51) Int. Cl. C12N 1/20; C12M 1/107

(52) U.S. Cl. 435/243; 435/295.1

(57) ABSTRACT

A biogas system includes a fermenter having a first fermenting chamber and a second fermenting chamber for the fermentation of a fermenting medium. Biogas formed in the first fermenting chamber can be introduced into a riser pipe disposed in the second fermenting chamber.

The present invention relates to a, biogas system with a fermenter which has a first and at least a second fermenting chamber for the fermentation of the fermenting medium.

The invention also relates to a method of mixing fermenting medium in a fermenter of the afore-described kind.

In fermenting chambers it is possible to produce energy-rich biogas from organic substances, e.g. sewage sludge, liquid manure, vegetable waste, plant clippings and other agricultural waste material. This biogas can be converted into heat and electrical energy in machines like gas engines and turbines. With the liberalisation of the Austrian gas market it became possible for biogenic gas producers to supply the public natural gas network, provided that the prescribed quality requirements were observed. A prerequisite for rapid fermentation and effective biogas production is homogeneous thorough mixing of the fermenting medium in the fermenting chambers, so that the solid material in the fermenting medium is not deposited on the floor, but continues to be in suspension. Mechanical mixing systems, e.g. slow rotating paddle mixers with a vertical axis, or fast rotating propeller mixers, amongst others, are prior art mixing systems for biogas systems. These mixing systems, in addition to high manufacturing costs, also have the drawback that they require intensive maintenance.

It is therefore an object of the present invention to propose a biogas system of the kind mentioned in the introduction, in which there is homogeneous thorough mixing of the fermenting medium, but wherein the use of mechanical mixers is not absolutely necessary.

This is achieved according to the invention in one advantageous embodiment by virtue of the fact that biogas formed in the first fermenting chamber can be introduced into a riser pipe disposed in the second fermenting chamber.

In this way, a means is created for the largely, anaerobic decomposition of organic substrates by utilising the gas pressure of the biogas which is produced in the first fermenting chamber and which is able to be forced into the riser pipe of the second fermenting chamber. The riser pipe—preferably extending over at least most of the maximum height of the fermenting chamber—of the second fermenting chamber rests on the basic principle of a mammoth pump where intake of biogas gives rise to a mixture of fermenting medium and biogas of significantly lower specific weight than the fermenting medium surrounding the riser pipe. In other words, the rising gas bubbles in the riser pipe of the second fermenting chamber reduce the density of the liquid in comparison with the surrounding liquid. The difference in density causes an ascending flow in the riser pipe which thus serves for circulation around the reactor.

In a preferred embodiment of the invention, it is provided that the riser pipe is designed in such a way that the fermenting medium reaches the riser pipe through an intake opening, and issues back out of the riser pipe through a discharge opening. In this connection, it is provided that the intake opening is displaced below the discharge opening. For an optimum fermentation process it can be advantageous if the riser pipe is disposed in the fermenting chamber substantially vertically and preferably centrally therein.

In order that even a relatively small gas pressure of the produced biomass is sufficient to overcome the hydrostatic pressure of the column of liquid, and thus induce an ascending flow in the riser pipe, it can be advantageous if the biogas can be introduced in the lower region, preferably in the bottom-most half, of the uppermost third of the riser pipe.

According to a preferred embodiment of the invention it can be provided that the riser pipe has a heating means, preferably a heat exchanger. In this way, the reactor can be heated, and as a result of the increased temperature of the liquid in the riser pipe an additional difference in density is produced in comparison with the liquid in the surrounding reactor space. In this connection, it can be advantageous if the riser pipe is designed so that it is double-walled at least regionally, wherein a heating fluid, preferably heating water, can circulate between the two walls.

The heating water can, for example, be supplied to the riser pipe by way of the excess heat of a block-type thermal power station. Good heat transfer can also be achieved because of the resultant improved mixing flow.

According to one embodiment of the invention, it can be provided that the biogas is transferred from the first fermenting chamber. into the riser pipe of the second fermenting chamber via a gas pipe which is preferably closed apart from one intake and one outlet. Of course, if necessary, gas valves can also be used which permit gas to be conveyed into the riser pipe of the second fermenting chamber when a set, or presettable, (excess)-pressure prevails in the first fermenting chamber.

Advantageously, it is provided that the first fermenting chamber is designed to be gas-tight, at least in the filled condition—except for the gas pipe. In this way, the necessary gas pressure can be prepared in the gas tower of the first fermenting chamber.

According to a further embodiment of the invention, it can be provided that a riser pipe is likewise disposed in the first fermenting chamber. Therein, the riser pipe can have all of the features which have been described for the riser pipe of the second fermenting chamber. In this connection, it can be advantageous if compressed air can be introduced into the riser pipe of the first fermenting chamber, so that advantageously the biogas can be desulfurized.

The method according to the invention of mixing fermenting medium in a fermenter which has a first and at least a second fermenting chamber for the fermentation of the fermenting medium is characterized in that biogas formed in the first fermenting chamber is introduced into a riser pipe disposed in the second fermenting chamber in order to produce a flow therein. In this connection, it is advantageous if the riser pipe is heated.

Further details and advantages of the present invention will be described with the aid of the following description of the drawings, wherein:

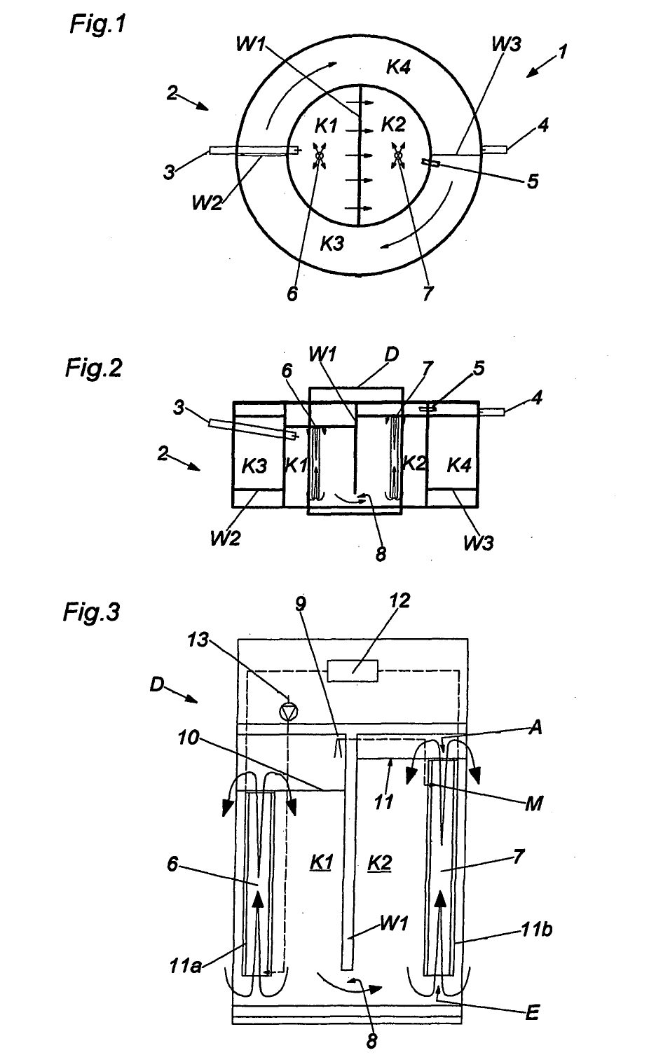

FIG. 1 is a diagrammatic section through a biogas system according to the invention in a top plan view,

FIG. 2 is a vertical section through the biogas system of FIG. 1, and

FIG. 3 is a detail, shown on a larger scale, of the fermenting chamber of FIG. 2.

FIG. 1 is a diagrammatic view in plan of a biogas system 1 according to the invention. This biogas system 1 comprises a fermenter 2 which is of a circular shape for reasons associated with statics, hydraulics and heating technology. The fermenter 2 contains a first fermenting chamber K1 and a second fermenting chamber K2 which jointly describe the form of a circle. The reference numeral 3 is used to denote an intake through which the fermenting medium, e.g. liquid manure, can be introduced into the fermenting chamber K1 from above. The fermenter 2 further comprises two post-fermenting chambers K3 and K4, wherein an outer wall which is formed by the first and second fermenting chambers is disposed essentially concentrically with respect to the outer wall of the two post-fermenting chambers K3 and K4. The post-fermenting chamber K4 has an outlet 4 for liquid. The fermenting chambers K1 and K2 thus form the core, and the post-fermenting chambers K3 and K4 form the circular periphery, wherein the individual fermenting chambers K1, K2, K3, K4 are separated from each other by walls W1, W2, W3, preferably downflow baffles, so that in the region of the fermenter 2 close to the floor, it is possible for fermenting medium to pass from one fermenting chamber into the other, as shown in FIG. 2. There is a clear hydraulic division between the core and periphery (e.g. between chambers K2 and K3), where there is merely an overflow opening 5 at water level. In the embodiment shown, a riser pipe 6 is disposed in the first fermenting chamber K1, and a riser pipe 7 is disposed in the second fermenting chamber K2. Now, the invention rests on the basic concept of introducing the biogas occurring as a result of the fermenting process in the first fermenting chamber K1 into a riser pipe 7 disposed in the second fermenting chamber K2, so as to induce an ascending flow therein. This ascending flow serves to provide circulation around the reactor, so that solid matter is not deposited on the bottom of the fermenter 2, but continues to be in suspension, thereby producing homogeneous decomposition of the substrate. A riser pipe 6 with the features described within the context of this invention can likewise be disposed in the first fermenting chamber K1, but compressed air is forced into the riser pipe 6, instead of biogas into the Thermo-Gas-Lift (a part of ca. 4% air has proven advantageous for sulfur-free biogas).

FIG. 2 shows a vertical section through the fermenter 2 with both fermenting chambers K1 and K2 which are separated from each other by a downflow baffle W1, so that the first fermenting chamber K1—except for the gas pipe [FIG. 3] leading to the riser pipe 7 of the second fermenting chamber K2—is designed in such a way that it is gas-tight at the top, and, at the bottom, permits passage of the fermenting medium from one fermenting chamber into the other (or, in the opposite direction). To that end, the downflow baffle W1 has in the bottom-most region, preferably in the bottom-most quarter, a gap 8 which extends across the width of the downflow baffle W1, through which gap the fermenting medium is able to flow. The downflow baffles W2 and W3 are designed in a similar way. The reference letter D is used to denote a cut-out detail which is shown on a larger scale in FIG. 3.

FIG. 3 is the cut-out detail D of FIG. 2, on a larger scale, with reference to which the operating principle of the fermenter 2 according to the invention will now be described more closely. The riser pipe 7 is designed in such a way that the fermenting medium arrives at the riser pipe 7 through an intake opening E, and issues back out of the riser pipe 7 at a discharge location A located at a higher level.

The purpose of the downflow baffle W1 is to separate the two fermenting chambers K1 and K2, wherein the downflow baffle W1 has a closed wall in the upper region and a gap 8 in the lower region. As a result of the intense production of biogas in the fermenting chamber K1, an excess pressure builds up in the gas-tight tower of the fermenting chamber K1, and forces down the level of liquid 10, and a corresponding volume of liquid is forced under the downflow baffle W1 and through into the fermenting chamber K2. The maximum level 11 in the fermenting chamber K2 is determined by the overflow opening 5 (FIG. 1, FIG. 2) into the post-fermenting chamber K3. A gas-overflow pipe 9 which goes from the fermenting chamber K1 is forced directly into the riser pipe 7 of the second fermenting chamber K2 at the entry location M. The height location of the entry location M defines the excess pressure in the fermenting chamber K1 and makes costly pressure gauging- and regulating instruments superfluous. The rising gas bubbles reduce the density of the liquid in the riser pipe 7 in comparison with the surrounding liquid in the fermenting chamber K2, thereby producing an upwardly directed vertical flow. The riser pipe 7 should open out only slightly below the maximum level 11. The difference in height between the entry location M of the biogas excess pressure line 9 into the riser pipe 7 and the overflow opening 5 defines the gas pressure in the first fermenting chamber K1. As soon as that gas pressure has built up as a result of the biological activity, the gas flows continuously, at a constant pressure, through the gas pipe 9 into the riser pipe 7 of the fermenting chamber K2. The rising gas bubbles accelerate the vertical flow of the liquid, and the gas which has escaped is able to overflow in almost pressure-free manner into the gas spaces of the post-fermenting chambers K3 and K4, and carry on flowing towards a gas accumulator disposed outside the fermenter 2. The effect of the vertical flow is yet further intensified by the heatable riser pipes 6 and 7, since these latter are equipped with a heating means 11a and 11b. In the embodiment shown, the heating means 11a and 11b are in the form of a heat exchanger, a heating fluid, preferably heating water from a tank 12 of heating water, being able to circulate between the two walls by virtue of the double-walled construction of the riser pipes 6 and 7. The tank 12 of heating water thus supplies both riser pipes 6 and 7, flow being promoted by the heat input, and heat transfer also being facilitated towards the contact surfaces around which it flows. The riser pipe 6 of the first fermenting chamber K1 has a supply 13 of compressed air, wherein the forcing of air into the riser pipe 6 of the first fermenting chamber K1 represents the starting point of de-sulfurized air being forced through the gas towers of all four fermenting chambers K1 to K4. As a result of the small amount of oxygen, microbial oxidation of the H₂—S-sulfur is possible on the surfaces of the tower, and by avoiding short circuit currents of biogas or air a degree of desulfurization in the pipe is ensured. Along the flow path through the gas towers of the four chambers K1-K4 sufficient reaction surfaces are available for H₂S-oxidation, and the elementary sulfur which has precipitated arrives back in the bio-liquid manure. Although the amount of compressed air is substantially less than the amount of pressurised gas produced from fermenting chamber K1, the introduction of compressed air can take place much lower down, i.e. at the lower opening of the riser pipe 6. Therefore, the Thermo-Gas-Lift in the fermenting chamber K1 attains a similar carrying capacity to that in fermenting chamber K2.

According to one embodiment of the invention it can be provided that the gas pipe 9 comprises an overflow valve—preferably capable of opening intermittently—by means of which the gas pressure in the first and second fermenting chambers K1, K2 can be equalized. This can, for example, be done by a by-pass line which branches off from the gas line 9, the gas being able to be introduced directly into the second fermenting chamber K2. The level of liquid of fermentation chamber K2 is pushed down by the prevailing gas pressure, whereupon a corresponding volume of liquid, starting from the second fermenting chamber K2, is urged through the gap 8 in the downflow baffle W1, into the first fermenting chamber K1. As a result, the layer of sludge on the floor of the two fermenting chambers K1, K2 begins to flow at increased speed, so that the substrate which is close to the floor is mobilised, at least intermittently, and solid matter is not able to become permanently deposited on the floor.

The proposed biogas system with its 4-chamber plan in this way gives rise to a so-called “plug flow” characteristic, i.e. contrary to a fully thorough-mixing reactor a minimum residence time of the substrate is ensured, and hydraulic short-circuits are avoided, thereby bringing about more complete decomposition (greater yield of biogas, better quality bio-liquid manure in terms of hygiene-related parameters and odorous substances). By virtue of the concentric arrangement of the four fermenting chambers (fermenting chambers K1 and K2 with the greatest conversion of gas in the core, post-fermenting chambers K3 and K4 at the periphery) and an optimum volume/surface ratio (>1), heat loss is minimised, and temperature gradients are made possible between core and periphery. Furthermore, the hydraulic decoupling of core and periphery (overflow of liquid manure and gas without reflux) means that a high level of volume flexibility is obtained. The afore-described mixing system feeds seeding sludge from fermenting chamber K2 into fermenting chamber K1, and the core can therefore be operated independently, i.e. fermenting chambers K3 and K4 can be used and emptied both in the manner of reaction volumes as well as in the manner of gas-tight end disposal units.

The present invention is not only limited to the embodiment shown, but encompasses or extends to all variants and technical equivalents which can come within the scope of the following claims. The positional information selected in the description, e.g. above, below, etc. referring to the conventional mounting orientation of the fermenter, or to the drawing which has been directly described and shown, can, in the event of positional changes, be applied to the new orientation accordingly. Passive mixing devices can also be provided, such as perforated grids, which are disposed in the region of the layer of scum of the fermenting medium in fermenting chambers K1 and K2. If there is equalization of pressure between fermenting chambers K1 and K2, as triggered by the overflow valve, the fermenting medium forces its way through the perforated grid, thereby preventing solidification of the layer of scum.

1. A biogas system comprising:
a fermenter having a first fermenting chamber and at least a second fermenting chamber for the fermentation of a fermenting medium;
wherein biogas formed in said first fermenting chamber can be introduced into a riser pipe disposed in said second fermenting chamber.

2. The biogas system according to claim 1, wherein said riser pipe is designed in such a way that said fermenting medium reaches said riser pipe through an intake opening, and issues back out of said riser pipe through a discharge opening.

3. The biogas system according to claim 2, wherein said intake opening is disposed below said discharge opening.

4. The biogas system according to claim 1, wherein said riser pipe is disposed in said second fermenting chamber substantially vertically.

5. The biogas system according to claim 1, wherein biogas can be introduced approximately in the lower region or in the bottom-most half, of the uppermost third of the riser pipe.

6. The biogas system according to claim 1, wherein said riser pipe has a heating means.

7. The biogas system according to claim 1, wherein said riser pipe is designed so that it is double-walled at least regionally, wherein a heating fluid or heating water is capable of circulating between the two walls of said riser pipe.

8. The biogas system according to claim 1, wherein said biogas is transferred from said first fermenting chamber into said riser pipe of said second fermenting chamber via a gas pipe.

9. The biogas according to claim 8, wherein said gas pipe preferably closed apart from an intake and an outlet for said biogas.

10. The biogas system according to claim 8, wherein said first fermenting chamber is designed to be gas-tight—at least in a filled condition of said first fermenting chamber—except for said gas pipe.

11. The biogas system according to claim 8, wherein said gas pipe comprises an overflow valve which can at least be opened intermittently, by means of which a gas pressure between said first fermenting chamber and said second fermenting chamber can be equalized.

12. The biogas system according to claim 8, wherein said first fermenting chamber and said second fermenting chambers are separated from each. other by a wall, so that said first fermenting chamber—except for said gas pipe—is designed to be gas-tight at the top, and, at the bottom, allows the fermenting medium to pass from one fermenting chamber into the other.

13. The biogas system according to claim 12, wherein said wall is in the form of a downflow baffle.

14. The biogas system according to claim 13, wherein said downflow baffle has an opening in the bottom-most region thereof, which is provided so that said fermenting medium passes from one fermenting chamber into the other.

15. The biogas system according to claim 14, wherein said opening is in the form of a gap extending across the width of said downflow baffle.

16. The biogas system according to claim 1, wherein a riser pipe is likewise disposed in said first fermenting chamber.

17. The biogas system according to claim 16, wherein compressed air can be introduced into said riser pipe of said first fermenting chamber.

18. The biogas system according to claim 1, wherein said first fermenting chamber and said second fermenting chamber jointly describe an essentially circular form.

19. The biogas system according to claim 1, wherein said fermenter comprises at least one post-fermenting chamber.

20. The biogas system according to claim 19, wherein said at least one post-fermenting chamber encloses said first fermenting chamber and said second fermenting chamber at least regionally or completely.

21. The biogas system according to claim 19, wherein said second fermenting chamber communicates with said post-fermenting chamber via an overflow opening for said fermenting medium.

22. The biogas system according to claim 19, wherein said post-fermenting chamber has at least two sectional chambers.

23. The biogas system according to claim 19, wherein an outer wall formed by said first fermenting chamber and said second fermenting chamber is disposed substantially concentrically with respect to said outer wall of said post-fermenting chamber.

24. A method of mixing fermenting medium, comprising:
a fermenter having a first fermenting chamber and at least a second fermenting chamber for the fermentation of a fermenting medium,
wherein biogas formed in said first fermenting chamber is introduced into a riser pipe disposed in said second fermenting chamber in order to produce a flow in said second fermenting chamber.

25. The method according to claim 24, wherein said riser pipe is heated.
